(19) United States
(12) Patent Application Publication
(10) Pub. No.: US 2026/0174764 A1
(43) Pub. Date: Jun. 25, 2026

(54) COMPOSITION AND METHOD FOR REMOVING PLAQUE FROM AN ARTERY

(71) Applicant: HERBARIUS LTD, Ramat Hasharon (IL)

(72) Inventor: Arnon KOREN, Ramat Hasharon (IL)

(21) Appl. No.: 19/110,409

(22) PCT Filed: Sep. 12, 2024
(86) PCT No.: PCT/IL2024/050921

§ 371 Date: Mar. 10, 2025

Related U.S. Application Data
(60) Provisional application No. 63/538,332, filed on Sep. 14, 2023.

(51) Int. Cl. A61K 31/519; A61B 17/12; A61K 9/00; A61K 31/44; A61K 38/00

(52) U.S. Cl. CPC A61K 31/519; A61B 17/12136; A61K 9/0019; A61K 9/0056; A61K 31/44; A61K 38/00

(57) ABSTRACT

The invention concerns methods and compositions comprising Chymopapain for dissolving atherosclerotic plaques disposed on an inner surface of an artery.

### TECHNOLOGICAL FIELD

The present disclosure generally relates to compositions comprising chymopapain and methods for removal of arterial plaques.

### BACKGROUND ART

References considered to be relevant as background to the presently disclosed subject matter are listed below:
1. Simmons, J. W., Fraser, R. D. (2005). The Rise and Fall of Chemonucleolysis. In: Kambin, P. (eds) Arthroscopic and Endoscopic Spinal Surgery. Humana Press, pp 351-358.
2. Fraser, R. D. (1982). Chymopapain for the treatment of intervertebral disc herniation. Spine 7 (6): p 608-612.
3. WO/1985004417.
4. U.S. Pat. No. 3,072,532.
5. U.S. Pat. No. 3,320,131.

Acknowledgement of the above references herein is not to be inferred as meaning that these are in any way relevant to the patentability of the presently disclosed subject matter.

### BACKGROUND

According to the Centers for Disease Control and Prevention, one person dies every 34 seconds in the U.S. from cardiovascular disease. Indeed, atherosclerosis associated complications such as ischemic stroke, cerebrovascular disease, peripheral arterial disease, and kidney disease sum up to be the leading cause of mortality in the Western countries. Atherosclerosis is a condition of stenosis and sclerosis of the arteries that develops when plaque builds up inside the arteries and blocks the bloodstream. The plaque develops slowly from cholesterol, fat, calcium, blood cells and other substances building up in the arteries' walls. Atherosclerosis is a multisystemic, chronic and progressive disease which develops slowly. The NIH estimates that about half of Americans between ages 45 and 84 have atherosclerosis and are not aware of it.

Presently, despite the increase in the public awareness for healthy lifestyle, lipid-lowering therapies and other prevention programs, Atherosclerosis is still at the top of the list of death causes. Currently, there is still no medicine available that directly treats atherosclerosis. There are medications for other diseases that are a risk factor for the development of atherosclerosis, such as diabetes, hypertension, and an unbalanced cholesterol level in the blood.

All the direct medical intervention solutions are invasive surgical operations such as stent placement and vascular surgery, for example: Percutaneous coronary intervention (PCI) followed by implanting a stent to prevent the artery from narrowing down; Coronary artery bypass grafting (CABG) which employs normal arteries from the chest wall or veins from the legs to bypass the blocked arteries; Trans-myocardial laser revascularization or coronary endarterectomy which treats severe angina associated with coronary heart disease; Carotid endarterectomy, angioplasty, or carotid artery stenting which treats carotid artery disease.

Chymopapain was first isolated in 1941 from the crude latex derived from Carica papaya. It is the main proteolytic component of the latex and is an extracellular plant cysteine proteinase. WO/1985004417 describes methods for commercial scale isolation of chymopapin B or C and provides a list of various medicinal uses for proteolytic enzymes derived from papaya including U.S. Pat. No. 3,072,532 (use in chemotherapy) and U.S. Pat. No. 3,320,131 (use for treatment of herniation). Chymopapain was administered as an intradiscal injection for the treatment of sciatica from lumbar disc protrusion, although it is no longer in use. [1, 2]. None of the background art documents above suggests the use of chymopapain for the treatment of atherosclerosis.

### GENERAL DESCRIPTION

In one aspect, the present invention provides a method of dissolving an atherosclerotic plaque disposed on an inner surface of an artery in a subject in need thereof, the method comprising administering to said subject a therapeutically effective amount of a pharmaceutical composition comprising Chymopapain.

In another aspect, the present invention provides a pharmaceutical composition for dissolving an atherosclerotic plaque disposed on an inner surface of an artery in a subject, wherein the pharmaceutical composition comprises chymopapain and one or more physiologically acceptable carriers.

In another aspect, the present invention provides chymopapain for use in dissolving an atherosclerotic plaque disposed on an inner surface of an artery in a subject.

In another aspect, the present invention provides a method of treating, preventing, or ameliorating atherosclerosis or an atherosclerosis associated complication, the method comprising administering to a subject in need thereof a therapeutically effective amount of a pharmaceutical composition comprising Chymopapain.

In another aspect, the present invention provides a pharmaceutical composition for treating, preventing, or ameliorating atherosclerosis or an atherosclerosis associated complication in a subject, wherein the pharmaceutical composition comprises chymopapain and one or more physiologically acceptable carriers.

In one embodiment, said subject is a human.

In one embodiment, said pharmaceutical composition is administered to an isolated portion of an artery comprising said atherosclerotic plaque.

In one embodiment, said administration is performed by flushing the isolated portion of the artery with the pharmaceutical composition.

In one embodiment, said method comprises bypassing said isolated portion with a temporary blood duct to maintain blood flow concurrent with said flushing.

In one embodiment, said artery is a limb artery and the administration is performed using isolated limb perfusion or isolated limb infusion.

In one embodiment, said artery is a coronary artery and the administration is performed by flushing a coronary artery with said pharmaceutical composition at the cardioplegia stage of a coronary surgery.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1 is a schematic illustration of a system for plaque treatment in an isolated intact artery using a temporary carotid shunt.

FIGS. 2A and 2B are schematic illustrations of a system for plaque treatment in an isolated intact artery using a temporary bypass. 2A—perfusion is performed using a pump; 2B—perfusion is performed using an infusion set.

FIG. 3 is a schematic illustration of a system for plaque treatment in an isolated limb artery.

FIGS. 4A and 4B are schematic illustrations of a system for treatment of atherosclerotic coronary artery during coronary surgery. 2A—perfusion is performed using a pump; 2B—perfusion is performed using an infusion set.

FIG. 5 is a photograph of an SDS-polyacrylamide gel. Lane 1: MW ladder, Lane 2: active compound isolated from the hydrophilic crude extract of latex derived from the fruit of Papaya Sp. The arrow indicates the band representing the isolated compound. The numbers on the left represent kD according to the MW ladder.

FIG. 6A-6B are pictures showing the effect of the isolated chymopapain of the invention on an isolated human atherosclerotic plaque tissue. The figures show results of a plaque treated for 4 days (+short vortex once a day) with the isolated chymopapain of the invention (Treated) and of an untreated plaque kept in saline alone (Control). FIG. 6A shows results of an experiment performed in a 96 plate well. FIG. 6B shows results of an experiment performed in an Eppendorf tube.

FIG. 7A-7C are pictures showing the effect of Chymopapain on a clogged human femoral artery. FIG. 7A shows the perfusion system. FIGS. 7B and 7C show the artery lumen (marked by arrows) before (B) and after (C) treatment with Chymopapain for 48 hours.

FIG. 8A-8B are pictures showing the structure of the artery wall after perfusion of the artery for 48 hours with Chymopapain. FIG. 8A is a cross section of the artery in a magnification of ×16. The area in the dotted rectangle is shown in a higher magnification of ×50 in FIG. 8B.

FIG. 9 is a photograph showing results of a hemolysis assay. The figure shows a 96 well plate containing samples of human red blood cells that were washed and diluted to 20% with PBS and subjected to increasing concentrations of Chymopapain 0.125-8 mg/ml, no Chymopapain (0), or distilled water.

### DETAILED DESCRIPTION OF EMBODIMENTS

The present invention is based on the surprising finding that chymopapain was effective in dissolving arterial plaques, which are complex and hard to disassemble substances. As shown in the Examples below, chymopapain administration resulted in dissolution of human atherosclerotic plaques in vitro. Furthermore, perfusion of chymopapain into a portion of a blocked human femoral artery placed ex vivo in a closed loop system resulted in dissolution of the plaques from the inner surface of the clogged human artery.

In one aspect, the present invention thus provides a method of dissolving an atherosclerotic plaque disposed on an inner surface of an artery in a subject in need thereof, the method comprising administering to said subject a therapeutically effective amount of a pharmaceutical composition comprising Chymopapain.

As used herein the terms “dissolution” and “dissolving” refer to the erosion, dismantling, elimination or reduction in the size of an atherosclerotic plaque.

As used herein the term “Chymopapain” refers to a group of sulfhydryl proteolytic enzymes. Chymopapain was originally isolated from the latex of papaya (Carica papaya). It is a cysteine protease which belongs to the papain-like protease group. Chymopapain has a known proteolytic activity, but it may exert its biological activity of clearing arterial plaques through additional mechanisms, e.g., digestion of fat and/or sugars. As used herein, the term Chymopapain refers to both the isolated, plant derived forms of the enzyme as well as to a synthetically produced enzyme. The term encompasses all chymopapain isomers, including but not limited to Chymopapain A, Chymopapain B, and Chymopapain C. Moreover, the term also encompasses analogs or derivatives of chymopapain which retain the biological activity of the compound as described herein. In one embodiment, the chymopapain is the enzyme designated EC 3.4.22.6. This enzyme has a Mw of about 27 kD. In another embodiment, the chymopapain is a plant-derived isolated form of the enzyme having a MW of about 20 kD. In a specific embodiment the chymopapain is a plant-derived isolated form of the enzyme which was isolated by series of separations on column chromatography including SEC fractionation followed by several reverse-phase separations on c18 columns with increasing gradient concentrations of Acetonitrile in Water, e.g., according to the procedure described in the Examples below.

Chymopapain can be extracted from the plant latex using any method known in the art, for example as described in WO 85/04417, Monti et al (2000) (Brazilian Archives of Biology and Technology 43 (5): 501-507), in Buttle and Barrett (1984) (The Biochemical Journal. 223 (1): 81-88), or in Baines and Brocklehurst (1979) (The Biochemical Journal. 177 (2): 541-548). The latex may be taken from any plant part, for example the plant's fruit, leaves, stalk, stems or trunk.

A non-limiting example of an isolation procedure is described in the Examples below.

As shown in the Examples below, Chymopapain caused a dissolution of arterial plaques in vitro and opened a plaque blockage in a femoral vessel, while no detrimental effect on blood vessel walls and on erythrocytes was observed.

Accordingly, the present invention also provides a pharmaceutical composition for dissolving an atherosclerotic plaque disposed on an inner surface of an artery in a subject, wherein the pharmaceutical composition comprises chymopapain and one or more physiologically acceptable carriers.

The pharmaceutical composition of the invention aims at dissolving arterial plaques while leaving the blood vessel walls and blood cells intact and unharmed.

In another embodiment, the present invention provides a pharmaceutical composition for the prevention and treatment of atherosclerosis, and atherosclerosis associated complications, as well as for preventing the reoccurrence of the plaque and sedimentation of the internal arteries walls, wherein the pharmaceutical composition comprises chymopapain and one or more physiologically acceptable carriers.

As used herein the term “pharmaceutical composition” refers to a composition comprising Chymopapain, wherein said composition is provided as a medicament.

In some embodiments, the invention also encompasses nutritional compositions. As used herein the term “nutritional composition” refers to a composition comprising Chymopapain, wherein said composition is provided as a food supplement, nutrition additive, a botanical drug, food, or beverage.

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, formulations are prepared by uniformly and intimately bringing into association the active ingredients of the invention with liquid carriers or finely divided solid carriers or both, and then, if necessary, shaping the product.

The term “pharmaceutically acceptable” and “physiologically acceptable” are used interchangeably herein to denote an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer to a material, such as a carrier, or diluent, which does not abrogate the biological activity or properties of the compound, and is relatively nontoxic, e.g., the material may be administered to an individual without causing undesirable biological effects or interacting in deleterious manner with any of the components of the composition in which it is contained.

“Pharmaceutically acceptable excipient” as used herein, refers to any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, or preservatives used in formulating pharmaceutical products.

The compositions may be formulated into any of many possible dosage forms such as, but not limited to, powder, tablets, capsules, liquid syrups, nasal spray, nasal drops, soft gels, suppositories, patches, and enemas. The compositions may also include other agents conventional in the art having regard to the type of formulation in question.

The composition of the invention can be administered alone, or in combination with other active agent(s). In certain embodiments the additional active agents include, but are not limited to, statins and blood thinning medicaments.

The compositions of the present invention may be combined with other types of treatments including behavioral therapy, diet restrictions and pharmacological intervention.

In another aspect, the present invention provides a method of treating, preventing, or ameliorating atherosclerosis or an atherosclerosis associated complication, the method comprising administering to a subject in need thereof a therapeutically effective amount of Chymopapain or a composition comprising Chymopapain.

The terms “treating”, “preventing”, or “ameliorating” are used conventionally and refer to the management or care of a subject for the purpose of combating, alleviating, reducing, relieving, or improving a subject's atherosclerosis, any symptom thereof including any reduction in the plaque size, or an atherosclerosis associated complication. The term also encompasses prophylactic treatment of subjects at risk of atherosclerosis, as well as prevention of plaque reoccurrence.

As used herein the term “atherosclerosis” refers to thickening or hardening of the arteries, caused by a buildup of plaque in the inner lining of an artery; plaque is also referred to herein as “atherosclerotic plaque” or “arterial plaque”. Plaque is made up of deposits of fatty substances, cholesterol, cellular waste products, calcium, and fibrin. As it builds up in the arteries, the artery walls become thickened and stiff.

In some embodiments, the atherosclerosis associated complication is selected from a group consisting of coronary artery disease (CAD), peripheral artery disease (PAD), peripheral vascular disease (PVD), cerebrovascular disease, stroke, chronic kidney disease (CKD) caused by atherosclerosis, end-stage kidney disease (ESKD) caused by atherosclerosis, and acute kidney failure caused by atherosclerosis.

Cardiovascular diseases are often caused by or associated with atherosclerosis. As used herein the term “cardiovascular disease” refers to a group of disorders of the heart and blood vessels and includes coronary heart disease (CHD), congestive heart failure (CHF), hypertension, cerebrovascular disease, heart attacks and strokes, ischemic legs and gangrene, and chronic renal failure. Accordingly, in an embodiment, the pharmaceutical composition of the invention is used for treating a cardiovascular disease.

As used herein the term “artery” refers to a blood vessels present throughout the body that consists of a multi-layered artery wall wrapped into a tube-shaped channel. The present invention is suitable for the treatment of any type of artery. For example, but not limited to limb (leg and/or arm) artery, coronary artery, carotid artery, or renal artery.

The pharmaceutical composition of the invention can be administered and dosed in accordance with good medical practice. For example, the pharmaceutical composition can be introduced to the body by any suitable route which is effective to achieve the desired result including intravenous, intraarterial, oral, intraperitoneal, subcutaneous, transcutaneous, or intramuscular administration. The compositions may be administered systemically or may be locally administered. Local administration may be facilitated by using an implant that acts to retain the active dose at the site of implantation. The active agent may be formulated for immediate activity, or it may be formulated for sustained release.

In an embodiment, the pharmaceutical composition is administered by intravenous or intraarterial administration.

In one embodiment, the pharmaceutical compositions of the invention are administered to an isolated portion of an artery comprising an atherosclerotic plaque providing a limited regional treatment of the affected area. The pharmaceutical composition comprising chymopapain is flushed through the isolated portion of the artery and may be recycled one or more times as required. The isolation of an artery portion for regional chymopapain administration or flushing can be performed using any method known in the art, for example, but not limited to:

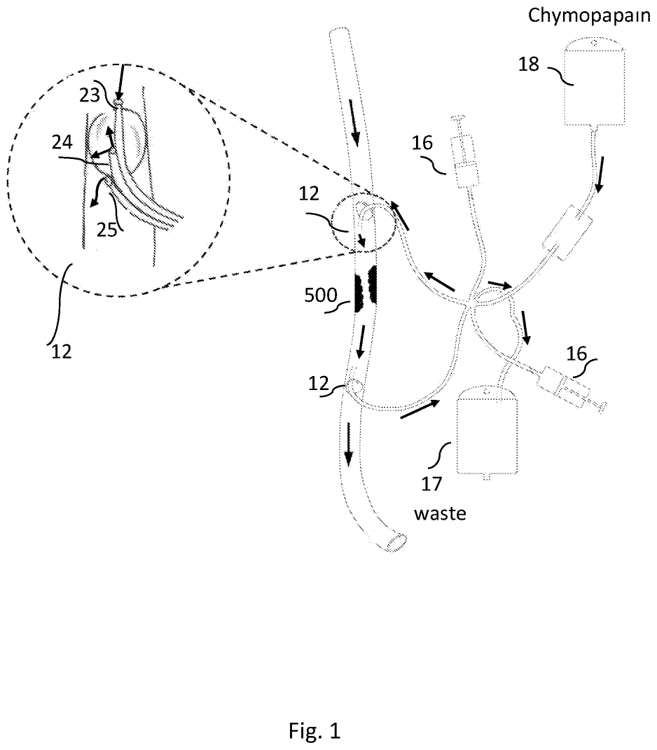

(i) A temporary vascular shunt (e.g., LeMaitre's Pruitt F3 or Flexcel temporary carotid bypass). Referring now to FIG. 1, schematically illustrating an exemplary embodiment of a system using a temporary intraoperative bypass to limit the blood flow in a segment of an artery 500 with plaque stenosis. The system comprises two occlusion balloons 12 inserted into the artery at the indicated points, and three channels (tubes) that pass through the occlusion balloon: the bypass channel 23, a channel for inflating the balloon 24 and the chymopapain infusion channel 25. The system further comprises one or more inflator syringes 16 for inflating the occlusion balloon, a waste bag 17 and an infusion bag 18 for supplying chymopapain. Arrows indicated the direction of flow. With this technique, a segment of an artery comprising the atherosclerotic plaque is blocked. The vascular shunt may be secured in the vessel using occlusion balloons. The restricted area which is separated from the systemic blood flow is perfused with chymopapain to dissolve the plaque. After the operation, the balloons are opened, and the blood flow is resumed.

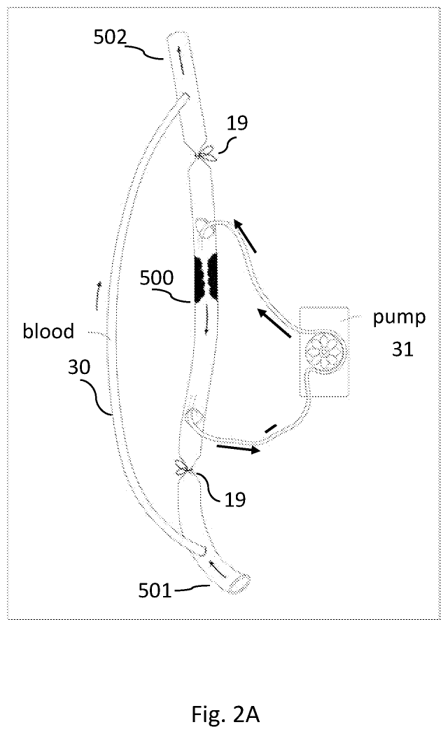

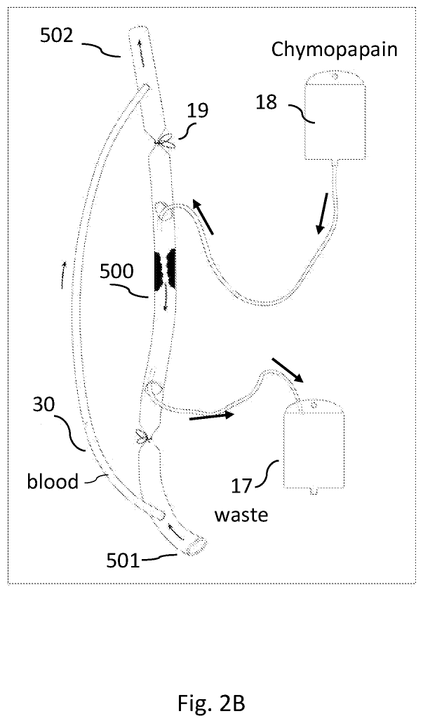

(ii) In patients suffering from injury involving both orthopedic and vascular trauma, a temporary intraoperative bypass is performed. Such a temporary bypass can be used to treat an organ affected by atherosclerosis without impeding the blood supply. Referring now to FIG. 2, schematically illustrating an exemplary embodiment of a system for performing occlusion and a bypass using a temporary bypass channel 30 (e.g., a plastic tube) to preserve the blood supply from a proximal segment 501 to a distal segment 502 of an artery 500 with plaque stenosis. The system comprises two occlusion elements 19 which block the blood supply to the afflicted artery segment, and a bypass channel 30 that allows for the blood to follow. In accordance with one embodiment depicted in FIG. 2A, the system further comprises a perfusion pump 31 for delivering and perfusing the chymopapain solution through the occluded artery portion. The chymopapain solution is introduced into the pump's tubing via a syringe. The pump comprises a filter (not shown in the figure) for removal of debris from the circulation. This is a closed system whereby the plaque is repeatedly exposed to the same chymopapain solution. In accordance with another embodiment depicted in FIG. 2B, instead of a pump, the system further comprises an infusion vessel 18 and a waste vessel 17, and the administration of chymopapain is performed gravimetrically by infusion. This is an open-flow system, whereby the plaque is exposed to a fresh chymopapain solution, which is not recycled within the system.

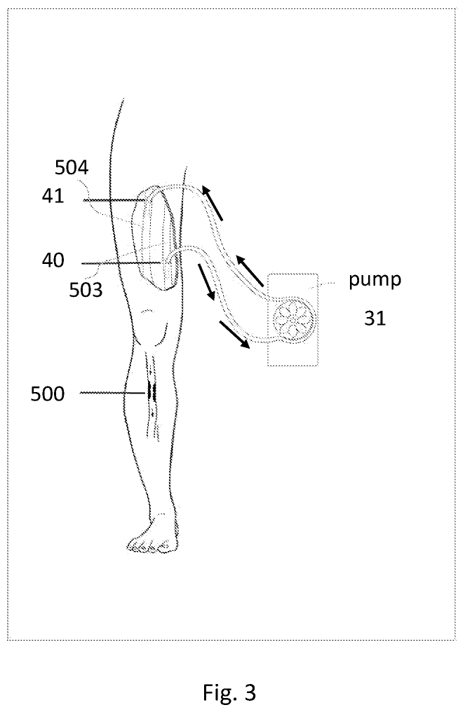

(iii) Isolated Limb Perfusion (ILP) or Isolated Limb Infusion (ILI). ILP or ILI are surgical procedures suitable for regional delivery of chymopapain into an artery in a limb (arm or leg). The limb is isolated from the rest of the body's blood supply and chymopapain may be distributed directly into the affected artery. Referring now to FIG. 3, schematically illustrating an exemplary embodiment of a system and a method for treatment of arterial plaque in an isolated portion of a limb artery 500 by perfusion. The system comprises an afferent catheter 40 configured to be inserted into the femoral artery 503 and an efferent catheter 41 configured to be inserted into the femoral vein 504. In this closed system, the limb (e.g., a leg) is treated with chymopapain solution which is perfused through the limb blood vessels using a perfusion pump 31 thereby exposing the atherosclerotic blockages 500 in the femoral, popliteal and tibial arteries to chymopapain. The pump comprises a filter (not shown in the figure) for removal of debris from the circulation.

Generally, the procedure comprises the following steps:
1. Tubes are placed into the feeding artery and draining vein of the limb, and a tourniquet is placed on the top of the limb to disconnect the blood supply of the limb from the rest of the body for a short time.
2. The tubes are then connected to a pumping machine that adds oxygen and chymopapain to the feeding artery and pumps it into the limb.
3. The blood and chymopapain are pumped around the limb for the required treatment period to dissolve the plaque (e.g., for about an hour) and then washed out.
4. The tubes are removed, the artery and vein are stitched up, and regular blood flow is resumed.

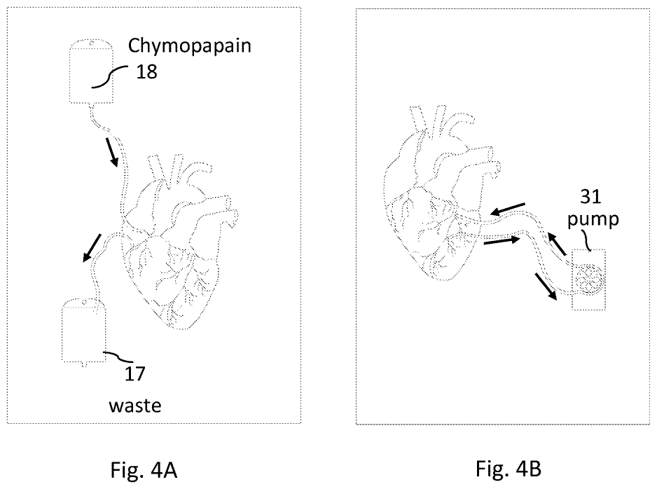

(iv) Flushing a coronary artery with chymopapain solution during coronary surgery at the cardioplegia stage. Referring now to FIG. 4, schematically illustrating an exemplary embodiment of a system and a method for treatment of an atherosclerotic coronary artery during the cardioplegia stage of coronary surgery. In accordance with an embodiment depicted in FIG. 4A, the system comprises an infusion vessel 18 and a waste vessel 17, and the administration of chymopapain is performed gravimetrically by infusion. This is an open-flow system, whereby the plaque in the coronary artery is exposed to a fresh chymopapain solution, which is not recycled within the system. In accordance with another embodiment depicted in FIG. 4B, the system comprises a perfusion pump 31 for delivering and perfusing the chymopapain solution through the occluded coronary artery. The chymopapain solution is introduced into the pump's tubing via a syringe. The pump comprises a filter (not shown in the figure) for removal of debris from the circulation. This is a closed system whereby the plaque in the coronary artery is repeatedly exposed to the same chymopapain solution. As shown in Example 3 below, flushing a chymopapain solution through a clogged artery resulted in dissolution of the plaque. According to a specific embodiment, the same principle is applied in vivo, namely flushing a chymopapain solution through a clogged coronary artery during cardioplegia.

Coronary bypass surgery is an operation performed to overcome narrowing or blockage of heart arteries by creating bypasses using thoracic arteries. Coronary bypass surgery is usually performed in hypothermia, which allows for 2-4 hours of treatment without damaging the heart or brain. The body is kept alive during this period by a heart-lung pump.

During coronary surgery the heart is arrested or stopped so that surgical procedures can be done in a still and bloodless field. This arrest is brought about by using a solution termed “cardioplegia”.

According to the present disclosure, instead of creating a bypass to resolve the narrowing or occlusion of the blood vessel, the blood vessel is flushed with a chymopapain solution during the cardioplegia period, while the solution and plaque debris are drained through the coronary sinus. This technique removes plaque from the arteries rather than bypassing them.

In an embodiment, the rate of plaque dissolution can be monitored either during or after the flushing of the artery (for example using duplex ultrasound). Duplex ultrasound can be used to assess the size of the plaque as well as the change (improvement) in blood circulation in the constricted area of the artery. The flushing of the artery procedure may be repeated one or more times as desired to achieve a sufficient reduction in the size of the plaque, complete elimination of the plaque, and/or improvement in the blood circulation in the affected portion of the artery.

### EXAMPLES

### Example 1

Preparation of the Active Chymopapains from Extracted Latex of Papaya Sp.

The chymopapain was isolated from hydrophilic crude extract of latex derived from the fruit of Papaya Sp. Through series of separations by column chromatography including SEC fractionation followed by several reverse-phase separations on c18 columns with raising gradient concentrations of Acetonitrile in Water.

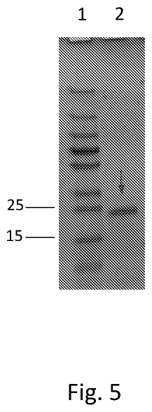

A 4% to 20% gradient SDS-polyacrylamide gel (Bio-Rad Laboratories Mini-Protean TGX precast gels) was prepared according to Manufacturer's instructions. As shown in FIG. 5, running the isolated compound on the SDS gel revealed that it has a molecular weight of about 20-25 kDa.

The isolated active compound was then subjected to proteomic analysis with LC-mass spectrometry (LC MS/MS) according to the following protocol:

### Proteolysis

The samples were suspended in 9M Urea, 400 mM Ammonium bicarbonate, 10 mM DTT and then incubated for 30′ at 60° C. (shaking) for full protein reduction. Then they were modified with 38 mM iodoacetamide (in the dark, room temperature for 30 min) and digested with modified trypsin (Promega) at a 1:50 enzyme-to-substrate ratio, overnight at 37° C. An additional second digestion was done for 4 hours.

### Mass Spectrometry Analysis

The tryptic peptides were desalted using C18 tips dried and re-suspended in 0.1% Formic acid.

The peptides (0.2 μg) were resolved by reverse-phase chromatography on 0.075×300-mm fused silica capillaries packed with Reprosil reversed phase material. The peptides were eluted with a linear 30 minutes gradient of 5 to 28% acetonitrile with 0.1% formic acid in water, 15 minutes gradient of 28 to 95% and 15 minutes at 95% acetonitrile with 0.1% formic acid in water at flow rates of 0.15 μl/min. Mass spectrometry was performed by Q Exactive plus mass spectrometer (Thermo) in a positive mode using repetitively full MS scan followed by collision induces dissociation (HCD) of the 10 most dominant ions selected from the first MS scan.

The mass-spec analysis revealed that the compound is a form of Chymopapain (EC 3.4.22.6) with a MW of ˜23000 Da. It is therefore referred to herein as “isolated chymopapain”.

### Example 2

### Dissolving the Plaque in an In Vitro Assay.

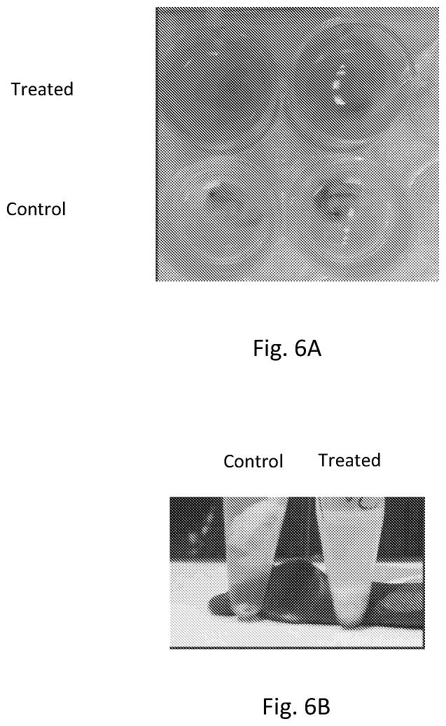

The isolated chymopapain (isolated as described in Example 1 above), at a concentration of 30 μg/ml, was added in-vitro to small fragments (2 mm)³ of human atherosclerotic plaque in saline solution and incubated for four days at room temperature. The specimens were vortexed for 20 seconds once a day. As shown in FIG. 6, the isolated chymopapain completely dismantled the plaques after 4 days.

### Example 3

### Opening a Plaque Blockage in the Femoral Vessel.

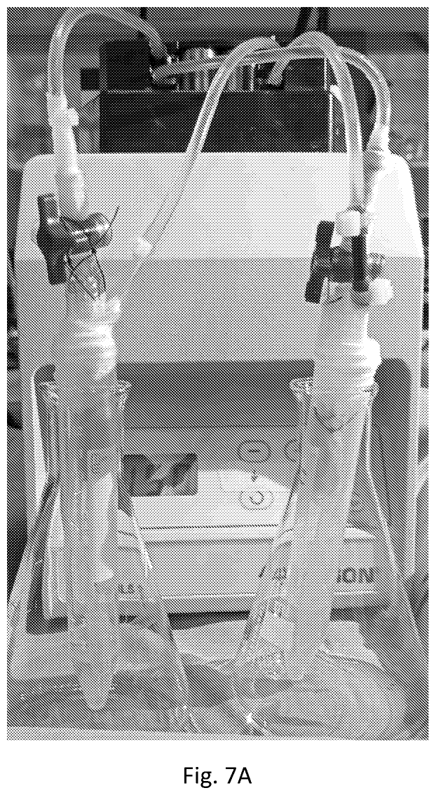

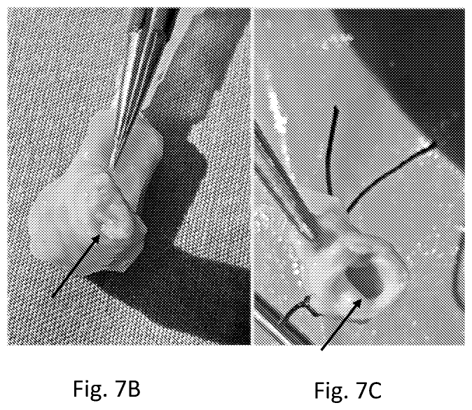

A portion of a blocked human femoral artery was cut transversely into two parts similar in dimensions and blockage level. Each part was connected in a closed loop of pipes and a peristaltic pump. One of the parts was subjected to perfusion (1 ml/min, at room temperature, for 2 days) with Chymopapain (3 mg/ml saline) and the other part served as a control and was perfused with saline under the same conditions. The test system is shown in FIG. 7A. As can be seen in FIGS. 7B and 7C, the isolated Chymopapain perfusion dissolved the plaques in the clogged human arteries in about 48 hours.

### Example 4

### Pathologic Examination.

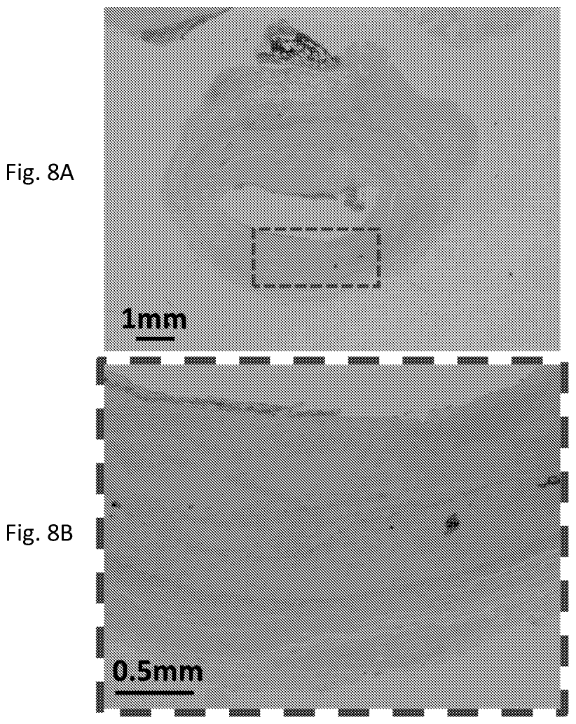

Since Chymopapain showed such efficacy in dissolving the plaque in clogged human arteries, it was important to verify whether this effect is specific to the plaque or whether Chymopapain causes any damage to the artery wall. Accordingly, slices of the artery, treated as described in Example 3 above, were fixed in PFA 4% for 24 h and stained with hematoxylin eosin. The specimen was then inspected under a microscope with magnification of ×16 and ×50. As can be seen in FIG. 8, the artery walls remained intact after the treatment, clearly indicating that the effect of Chymopapain is specific on the plaque and that it does not cause any damage to the artery wall.

### Example 5

### Hemolysis Assay.

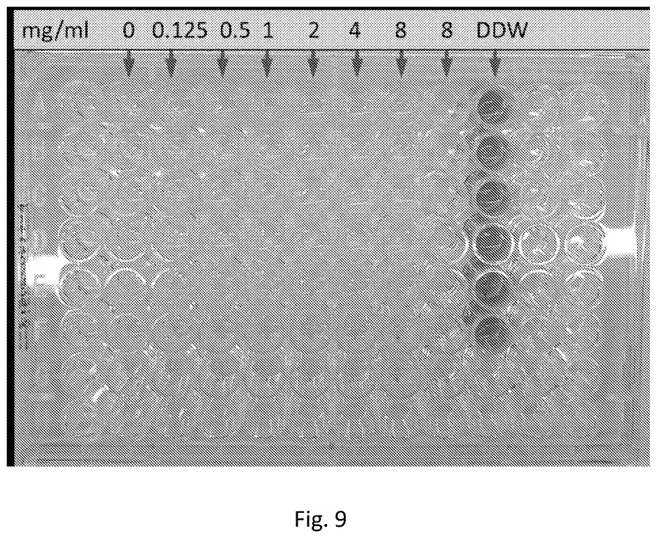

To verify that the perfusion of the artery with Chymopapain that caused the plaques to dissolve, has negligible side effects on the integrity of red blood cells from human blood, a Hemolysis assay was performed. Isolated human erythrocytes were washed and separated from the blood serum and were soaked in PBS (20% vol/vol). The erythrocytes were then incubated with series of Increasing concentrations of Chymopapain, ranging from 0.125 mg/ml to 8 mg/ml. The amount of hemoglobin that leaked out of the cells, colored the colorless PBS with its red color, and serves as an indication of the damage to the integrity of the cell membrane. The level of hemoglobin that leaked into the PBS was measured by a spectrophotometer (OD 600 nm). As demonstrated in FIG. 9, Chymopapain, even at the highest concentration, did not have any detectable hemolytic activity. The results were compared to the effect of DDW which causes maximal hemolysis of the erythrocytes and a maximal leakage of the hemoglobin into the medium.

1. A method of dissolving an atherosclerotic plaque disposed on an inner surface of an artery in a subject in need thereof, the method comprising administering to said subject a therapeutically effective amount of Chymopapain.

2. The method according to claim 1 wherein said subject is a human.

3. The method according to claim 1 wherein said Chymopapain is administered to an isolated portion of the artery comprising said atherosclerotic plaque.

4. The method according to claim 3, wherein said administering is performed by flushing the isolated portion of the artery with the Chymopapain.

5. The method according to claim 3 wherein said method comprises bypassing said isolated portion with a temporary blood duct to maintain blood flow concurrent with said flushing.

6. The method according to claim 3 wherein said artery is a limb artery and wherein said administering is performed using isolated limb perfusion or isolated limb infusion.

7. The method according to claim 3 wherein said artery is a coronary artery and wherein said administering is performed by flushing a coronary artery with said Chymopapain at a cardioplegia stage of a coronary surgery.

8. A pharmaceutical composition for dissolving an atherosclerotic plaque disposed on an inner surface of an artery in a subject, wherein the pharmaceutical composition comprises chymopapain, as a sole active agent, and one or more physiologically acceptable carriers.

9. (canceled)

10. A method of treating, preventing, or ameliorating atherosclerosis or an atherosclerosis associated complication, the method comprising administering to a subject in need thereof a therapeutically effective amount of a pharmaceutical composition comprising Chymopapain, as a sole active agent, and wherein said atherosclerosis associated complication is selected from a group consisting of coronary artery disease (CAD), peripheral artery disease (PAD), peripheral vascular disease (PVD), cerebrovascular disease, stroke, chronic kidney disease (CKD) caused by atherosclerosis, end-stage kidney disease (ESKD) caused by atherosclerosis, and acute kidney failure caused by atherosclerosis.

11. A pharmaceutical composition for treating, preventing, or ameliorating atherosclerosis or an atherosclerosis associated complication in a subject, the pharmaceutical composition comprises chymopapain, as a sole active agent, and one or more physiologically acceptable carriers, and wherein said atherosclerosis associated complication is selected from a group consisting of coronary artery disease (CAD), peripheral artery disease (PAD), peripheral vascular disease (PVD), cerebrovascular disease, stroke, chronic kidney disease (CKD) caused by atherosclerosis, end-stage kidney disease (ESKD) caused by atherosclerosis, and acute kidney failure caused by atherosclerosis.
